(19) United States
(12) Patent Application Publication
(10) Pub. No.: US 2016/0256130 A1
(43) Pub. Date: Sep. 8, 2016

(54) MONITORING STRUCTURAL FEATURES OF CEREBRAL BLOOD FLOW VELOCITY FOR DIAGNOSIS OF NEUROLOGICAL CONDITIONS

(71) Applicant: Neural Analytics Inc., Los Angeles, CA (US)

(72) Inventors: Robert B. Hamilton, Sherman Oaks, CA (US); Leo Petrossian, Los Angeles, CA (US); Dan Hanchey, Camarillo, CA (US)

(21) Appl. No.: 15/156,175

(22) Filed: May 16, 2016

Related U.S. Application Data
(63) Continuation of application No. PCT/US2014/065812, filed on Nov. 14, 2014.
Application No. PCT/US2014/065812 is a continuation of application No. 14/214,883, filed on Mar. 15, 2014, now abandoned.
(60) Provisional application No. 61/905,146, filed on Nov. 15, 2013.
(60) Provisional application No. 61/905,169, filed on Nov. 16, 2013.
(60) Provisional application No. 61/905,170, filed on Nov. 16, 2013.
(60) Provisional application No. 61/905,171, filed on Nov. 16, 2013.
(60) Provisional application No. 61/905,172, filed on Nov. 16, 2013.
(60) Provisional application No. 61/905,147, filed on Nov. 15, 2013.

(51) Int. Cl. A61B 8/06; A61B 5/00; A61B 5/055; A61B 8/08; A61B 8/00

(52) U.S. Cl. CPC A61B 8/06; A61B 8/488; A61B 8/4209; A61B 5/055; A61B 5/0042; A61B 5/0059; A61B 5/031

(57) ABSTRACT

The systems and methods described herein include a non-invasive diagnostic tool for intracranial hypertension (IH) detection and other neurological conditions like mild and moderate TBI that utilizes the transcranial Doppler (TCD) measurement of cerebral blood flow velocity (CBFV) in one or more cerebral vessels. A headset includes a TCD scanner which automatically locates various cerebral arteries and exerts an appropriate pressure on the head to acquire good CBFV signals.

### CROSS-REFERENCE TO RELATED APPLICATIONS

This application is a 35 U.S.C. §120 continuation of PCT/US2014/065812, with an International Filing Date of Nov. 14, 2014, which is a Continuation of U.S. Ser. No. 14/214,883, filed on Mar. 15, 2014; which in turn claims the benefit of the following U.S. Provisional Patent Applications: 61/798,645 filed on Mar. 15, 2013; 61/905,146 filed on Nov. 15, 2013; 61/905,147 filed on Nov. 15, 2013; 61/905,169 filed on Nov. 16, 2013; 61/905,170 filed on Nov. 16, 2013; 61/905,171 filed on Nov. 16, 2013; and 61/905,172 filed on Nov. 16, 2013; which are incorporated herein in their entirety.

### COPYRIGHT STATEMENT

A portion of the disclosure of this patent application document contains material that is subject to copyright protection including the drawings. The copyright owner has no objection to the facsimile reproduction by anyone of the patent document or the patent disclosure as it appears in the Patent and Trademark Office file or records, but otherwise reserves all copyright rights whatsoever.

### BACKGROUND OF THE INVENTION

1. Field of the Invention

The disclosure relates to the fields of physiological monitoring, and specifically to monitoring physiological functions of the brain, including intracranial pressure, cerebral blood flow velocity, cerebral blood flow, and cerebrovascular reserve. Acquisition of the physiological signals is performed by an automated ultrasound device for increased accuracy and reliability.

2. Description of the Prior Art

Neurological conditions including mild and severe traumatic brain injury (TBI), stroke or subarachnoid hemorrhage (SAH), cerebral malaria (CM), pseudotumor cerebri, and brain tumor affect millions of individuals worldwide each year. One specific physiologic parameter of interest is intracranial pressure (ICP), which is commonly defined as the pressure within cerebrospinal fluid (CSF) in the cerebral ventricles of the brain and is a critical parameter for managing brain injury patients because timely detection of acute ICP elevation is needed to guide treatment to prevent severe complications including cerebral ischemia and herniation. Unfortunately, the currently available clinical techniques for monitoring ICP and managing patients with risk of acute ICP elevation are invasive. For instance, one way to monitor intracranial pressure in the skull is with an intraventricular catheter which is introduced through a hole drilled through the skull and inserted into the lateral ventricle. Another invasive technique is to use a hollow subdural screw again inserted through a hole drilled in the skull and placed through the membrane that protects the brain and spinal cord (dura mater). Finally, a third invasive method is to insert an epidural sensor between the skull and dural tissue.

The invasive nature of ICP measurement obviates its application in many clinical circumstances where ICP measurements would be of significant diagnostic and prognostic value because of the increased risk of infection and secondary bleeding. One example is the management of acute liver failure patients. Since coagulopathy (bleeding disorder) is common among patients with acute liver failure, the risks associated with invasive ICP monitoring preclude its use, despite the significant potential benefits of outcome predictions based on measurements of elevated ICP. Another example is the diagnosis of idiopathic intracranial hypertension (IIH) aka pseudotumor cerebri, which would benefit from direct ICP measurements. Yet these measurements are rarely performed due to the associated risks and complexities of invasive ICP. Finally, CM provides another example of a condition which would benefit from ICP monitoring but because of the research limited areas where malaria is common it is technically infeasible.

Attempts have been made to identify reliable, non-invasive ICP monitoring techniques to meet these important unmet needs, but none of these attempts have demonstrated significant clinical applicability. Several groups have also proposed a few simple metrics of cerebral blood flow velocity (CBFV) such as systolic velocity, diastolic velocity, mean flow velocity, pulsatility index (PI), and resistance index for non-invasive assessment of ICP. It is, however, still controversial whether those simple metrics can provide reliable and accurate information about ICP.

In acknowledgment of the limitations of the current non-invasive ICP assessment techniques, improved systems and methods for increased ICP or intracranial hypertension (IH) detection can provide a significant benefit to patients and clinicians.

IIH is characterized by increased ICP of unknown cause and relatively common among obese young women. The management of IIH patients in the U.S. has been estimated to cost $444 million per year. Currently, IIH patients are treated with weight loss, medical therapy, and surgical therapy. Treatment decisions are often based on subjective symptoms, the presence and severity of papilledema, and invasive studies such as lumbar punctures. Given the variability of subjective symptoms and the possibility for papilledema to appear improved in the face of worsening disease if optic atrophy commences, a non-invasive IH diagnostic tool could simplify treatment decisions by allowing for real-time measurement of ICP and clinical correlation with changes in symptoms and signs. It could also improve patient outcomes by allowing earlier detection of changes in ICP followed by more efficient interventions to save vision in the face of worsening disease.

Another related but distinct physiologic deficit is that caused by mild TBI where there is no apparent increase in ICP but there remains a change in the underlying physiology (deficit in cerebrovascular reserve). Historically, the majority of research on mild TBI has focused on the neurological and neuropsychological outcomes of injury. Current diagnosis and return-to-play guidelines are largely based on results of neuropsychological tests that rely on patient symptoms such as the Post-Con Symptom Scale (PCSS), the Graded Symptom Checklist (GSC), the Standardized Assessment of Concussion (SAC), and Immediate Post-Concussion Assessment and Cognitive Testing (ImPACT). However, there is an unquestioned need to complement these neurological tests with methods that consider the pathophysiology of mild TBI. A recent review summarizes several pathophysiology-based methods to monitor mTBI, such as structural imaging (MRI, CT), diffusion tensor imaging, single photon emission CT, positron emission tomography, functional MRI, near-infrared spectroscopy, electroencephalography, magnetoencephalography, heart rate variability, and blood markers. However, the review highlights that most of these methods are in the early stages of research and that none has gained clinical acceptance.

As previously mentioned, a pathological increase in ICP is not present in mild TBI and therefore additionally physiological parameters need to be assessed. Several studies have identified changes cerebral hemodynamic changes following mild TBI, with a number of them investigating the possible root cause of the physiological deficit, a decrease in CBF. One related aspect of CBF is cerebrovascular reserve, the description of the range of cerebral perfusion variation from baseline. A change in this range of cerebral perfusion given a stimulus can be diagnostic/prognostic for a number of different conditions including: severe TBI, migraine, long-term spaceflight, stroke, and carotid artery stenosis. Cerebrovascular reserve can be assessed using non-invasive techniques including transcranial Doppler and therefore will benefit from the advanced framework purposed in this work.

### SUMMARY OF THE INVENTION

To date, traditional analysis of CBFV obtained using transcranial Doppler (TCD) has proven inadequate in the diagnosis of neurological conditions such as TBI and SAH. In acknowledgment of the limitations of current approaches for diagnosing TBI, it is thus desired to improved systems and methods for diagnosis of TBI and other neurological conditions.

The systems and methods described herein include collection of raw CBFV data from one or more blood vessels feeding the brain using transcranial Doppler (TCD), a system to combine and extract structural features using in-part, a database of previously validated CBFV pulses for the classification of various neurologic conditions including intracranial hypertension (IH) and mild/moderate TBI.

The systems and methods described herein include a non-invasive diagnostic tool for IH based on the structural analysis of CBFV waveforms measured via TCD. The performance of these systems and methods are validated by comparing two types of classification methods: one based on the traditional supervised learning approach and the other based on the semisupervised learning approach. Our simulation results demonstrate that the predictive accuracy (area under the curve) of the semisupervised IH detection method can be as high as 92% while that of the supervised IH detection method is only around 82%. It should be noted that the predictive accuracy based on traditional TCD features (pulsatility index (PI))-based IH detection method is as low as 59%.

TCD measurements may include the CBFV from one or more blood vessels in the head and neck. For example, measurements may be obtained from the middle cerebral artery (MCA), internal carotid artery (ICA), and/or basilar artery (BA), or any combination thereof.

In addition to the lack of accuracy of TCD caused by the limited feature set, inter- and intraobserver variation has plagued TCD adoption. To increase the reliability of our morphological framework we are also introducing a fully automated headset for the acquisition of the TCD signal.

In certain embodiments, the systems, devices, and methods include a method for non-invasively detecting IH. In certain approaches, this method includes detecting individual CBFV waveform pulses from a continuous CBFV segment, grouping the detected pulses, recognizing at least one valid pulse by utilizing a CBFV pulse library, constructing a representative pulse from the group, extracting over 100 structural features from the representative pulse, and using a classification framework to the ICP.

In certain approaches, the CBFV waveform segment is in association with a simultaneously recorded ECG segment. The method may further comprise identifying structural features including subpeaks of the constructed representative pulse. The method may include calculating representative metrics of the constructed representative pulse. For example, subpeak amplitudes may be used to characterize the ICP as normal or IH.

In certain embodiments, the systems and methods described herein include utilizing spectral regression for clustering the detected CBFV pulses. The methods may include constructing a graph by defining proper node connections. In certain approaches, the graph construction is weighted. In certain embodiments, the method includes decomposing eigenvectors. In certain approaches, regularized least squares are solved for at least one eigenvector. In certain embodiments, spectral regression includes kernel discriminant analysis. The systems and methods described herein provide for performing a decision a curve analysis by quantifying the predictive accuracy utilizing an area under the curve characteristic. In certain approaches, the intracranial pressure pulses are divided into three groups: normal (<15 mmHg), gray-zone (15-30 mmHg), and IH (>30 mmHg).

In certain embodiments, the systems and methods described could be used for the diagnosis of mild and moderate TBI where there is no increase in ICP. Our framework expands CBFV analysis from this rudimentary method to greater than 100 distinct structural features present in the waveform, thereby accurately quantifying subtle changes in the waveform and providing greater diagnostic and prognostic accuracy. A distinct advantage to our approach is that TCD-based devices are low-cost, safe, and portable and they have been shown to be effective in pre-hospital settings.

These and other embodiments are described in more detail herein. Variations and modifications of these embodiments will occur to those of skill in the art after reviewing this disclosure. The foregoing features and aspects may be implemented, in any combination and subcombinations (including multiple dependent combinations and subcombinations), with one or more other features described herein. The various features described or illustrated above, including any components thereof, may be combined or integrated in other systems. Moreover, certain structural features may be omitted or not implemented.

### BRIEF DESCRIPTION OF THE DRAWINGS

The foregoing and other objects and advantages will be apparent upon consideration of the following detailed description, taken in conjunction with the accompanying drawings, in which like reference characters refer to like parts throughout, and in which:

FIG. 1: Raw cerebral blood flow velocity (CBFV) data acquired from the TCD unit. The maximum velocity envelope is shown in white.

FIG. 2 Flow chart of the overall algorithm using multiple vessels collected using TCD from the head and neck.

FIG. 3: Block diagram of the structural feature extraction process showing a continuous CBFV input waveform that is transformed into one representative output CBFV pulse with three sub-peaks.

FIG. 4. Plots taken from Kim, S., et al., Noninvasive intracranial hypertension detection utilizing semisupervised learning. IEEE Trans Biomed Eng, 2013. 60(4): p. 1126-33.) show examples of CBFV waveforms associated with various mean ICP values: Top row (normal) and bottom row (hypertensive). Black dots represent three subpeaks. The CBFV waveforms associated with low mean ICP values (mICP in mmHG) tend to have more distinct subpeaks than those associated with high mean ICP pulses. The difference between the second and third subpeak amplitudes is greater in CBFV waveforms associated with high mean ICP pulses than it is in those associated with normal mean ICP pulses.

FIG. 5. A plot taken from Kim, S., et al. indicates AUC versus number of close neighbors (k), where each line and gray area represent the mean AUC and one standard deviation variation over multiple (=100) tenfold cross-validations.

FIG. 6. A plot taken from Kim, S., et al. shows an overall net benefit versus disease probability threshold pt, where the solid black line is for the Treat-All approach and the dotted black line for the Treat-None approach.

FIG. 7. A plot taken from Kim, S., et al. graphs continuous-scale label estimates of gray-zone samples versus corresponding ICP values as the results of the second cross-validation experiment, where the correlation coefficient between then was 0.55 with 2e-4 p-value.

FIG. 8. A plot taken from Kim, S., et al. illustrates ROC curve of the semisupervised²⁰⁰ IH detection method with three different operating points: the red dot is for the optimal accuracy operating point based on the Youden index with pₐ=0.12, the green dot is for the optimal net benefit operating point for pₜ=0.2, and the blue dot is for the optimal net benefit operating point for pₜ=0.4.

FIG. 9. Example of the major arteries of the cerebral circulation and the Circle of Willis.

FIG. 10. Front view of the portable transcranial Doppler device. The portable device will work with either hand and the screen will adjust to the given direction. The ultrasound probe is stored in the back magnetically.

FIG. 11. Rear view of the portable transcranial Doppler (TCD) device. The ultrasound probe is shown in its housing on the left.

FIGS. 12, 12A and 12B: Automated TCD headset design. Indication is shown on the front of the device. The dual ultrasound probes are contained in the side units of the device and will auto locate the MCA, ACA, and PCA based on a robotic system supplemented with a known database of vessel locations through the temporal window. FIGS. 12A and 12B are images of the exemplary TCD headset on the cranium of a patient.

FIG. 13 is a side view of another exemplary TCD headset worn by a patient having straps around the head and including a reciprocating scanner.

FIG. 14A is a perspective view of another TCD headset secured by anchors on the side of a patient's head with an outer housing in phantom to visualize internal components of the headset, and indicating adjustability for different sizes of patients, while FIG. 14B shows the outer housing against a profile of the wearer's head.

FIG. 15A is a side elevational views of the TCD headset of FIGS. 14A and 14B, and FIG. 15B shows the headset against a profile of the wearer's head to visualize components thereof.

### DETAILED DESCRIPTION OF THE PREFERRED EMBODIMENT

To provide an overall understanding of the systems, devices, and methods described herein, certain illustrative embodiments will be described. Although the embodiments and features described herein are specifically described for use in connection with monitoring intracranial pressure using transcranial Doppler (TCD) systems, it will be understood that all the methods, components, mechanisms, adjustable systems, manufacturing methods, and other features outlined below may be combined with one another in any suitable manner and may be adapted and applied to monitoring other physiological and nonphysiological characteristics including mild and moderate TBI using other types of non-invasive physiological monitoring including MRI and CT.

The term “non-invasive” pertains to methods of physiological monitoring that do not require surgery, or puncture wounds of any kind. As mentioned, in addition to a transcranial Doppler (TCD) system, an MRI system, a CT scanner, a pressure transducer, an optical imager, a near-infrared imager and other such devices are possible sources of raw data, and the application should be considered limited only by the appended claims.

The present application describes systems and methods for non-invasive collection of raw cerebral blood flow velocity (CBFV) data from one or more blood vessels feeding the brain as well as techniques to identify structural features in the CBFV waveform and extract those features for analysis. In this sense, “structural features” refers to identifiable characteristics (e.g., subpeaks, subtroughs, landmarks) of the measured CBFV waveform. As will be explained, these structural features can then be compared with previously identified reference data to classify the structural features and recommend a diagnosis.

The systems and methods described herein provide a non-invasive IH detection method based on the TCD measurement of CBFV in one or more blood vessels in the head and neck including the middle cerebral artery, internal carotid artery, basilar artery, vertebral artery, anterior cerebral artery, and other vessels that make up the Circle of Willis. These systems and methods are further enabled and demonstrated through example using various learning/classification algorithms.

For convenience, the following abbreviations are used throughout the text and description included herein:

aSAH—aneurysmal subarachnoid hemorrhage

ACA—anterior cerebral artery

AUC—area under the curve

BA—basilar artery

CBFV—cerebral blood flow velocity

ECG—electrocardiogram

ICA—internal carotid artery

ICP—intracranial pressure

IH—intracranial hypertension

IIH—idiopathic intracranial hypertension

MCA—middle cerebral artery

mTBI—mild traumatic brain injury

NPH—normal pressure hydrocephalus

PI—pulsatility index

ROC—receiver operating characteristic

SRKDA—spectral regression kernel discriminant analysis

TBI—traumatic brain injury

TCD—transcranial Doppler

The systems and methods described herein utilize an advanced, comprehensive structural feature analysis of CBFV waveforms for establishing alternative diagnostic methods for non-invasive ICP assessment and mild/moderate TBI.

IH detection is a classification problem to differentiate patients with elevated ICP from those with normal (non pathological) ICP. The traditional approach to such a classification problem is to use only labeled samples to train a given classifier, which is referred to as supervised learning. The major drawback of this approach is that it cannot utilize unlabeled samples even when useful information learned from them may result in the improvement of classification accuracy. Unlabeled samples may exist for various reasons such as the high cost or labor intensity of labeling all samples or the ambiguity in providing a binary label as in the case of IH detection and mild TBI/concussion diagnosis. For an example, a naïve approach would be to label CBFV waveforms as IH samples if the corresponding ICP is above 20 mmHg, which is a widely accepted threshold for considering ICP as elevated, and then to use a supervised learning algorithm to build the classifier. This straightforward paradigm may be too rigid making the detection of a true IH state critically dependent on the relevance of using 20 mmHg as a threshold, since for some patients categories an ICP level of 20 mmHg would not represented elevated levels (false positive) and for other patients a 20 mmHg threshold would miss an IH diagnosis. However, it is not an easy task to pick a different threshold, either. If the threshold is too high or too low, then one runs the risk of either missing IH diagnosis or creating too many false positives.

In order to address this ambiguity in labeling samples, the systems and methods described herein utilize a semisupervised learning classification approach. In the semisupervised learning approach, it is not necessary to label all samples since classifiers can be trained using both labeled and unlabeled samples. In certain approaches, the semisupervised learning techniques in the systems and methods described herein include generative models, self-training, co-training, transductive support vector machines, and graph-based methods. In certain approaches, ordinary regression techniques are combined with spectral graph analysis overcome several drawbacks of conventional graph-based semisupervised learning techniques.

In certain approaches, the systems and methods described herein are carried out using processing circuitry. As described herein, processing circuitry should be understood to mean circuitry, which includes one or more of a microcontroller, integrated circuit, application specific integrated circuit (ASIC), programmable logic device, field programmable gate array (FPGA), digital signal processors, application specific instruction-set processor (ASIP), or any other suitable digital or analog processors. This processing circuitry may be utilized as part of other user systems, including, but not limited to, computers, mobile devices, televisions, tablets, TCD monitoring systems, ECG monitoring systems, wearables or any other suitable device. Processing circuitry may be used to perform data and signal processing algorithms as described herein. Processing circuitry may be used to send and receive data, commands, user input to or from other network devices, included network connected systems and devices.

Processing circuitry may be coupled to electronic storage or memory. Electronic storage, as used herein, may include any appropriate readable memory media, including, but not limited to, RAM, ROM, EPROM, EEPROM, flash memory or other solid state memory technology, CD-ROM, DVD, or other optical storage, magnetic storage devices, or any other physical or material medium for storing desired information, data, instructions, software, firmware, drivers, or code. For example, storage may contain software instructions or machine code for controlling the input, output, and other processes of processing circuitry, such as performing algorithms and other process steps of the methods and systems described herein.

The processing circuitry may be part of a system which includes devices for interfacing with a user, such as a display and user input interface. For example, a display may be any suitable display interface, including, but not limited to a monitor, television, LED display, LCD display, projection, mobile device, headset, or any other suitable display system. A user input interface may be a keyboard, touchscreen, mouse, microphone, stylus, voice activated, or any other suitable user input interface. Displays and user input interfaces allow processing circuitry to provide information to the user and to receive user-generated commands, responses, and data. In certain approaches, the systems and methods described herein include actuators, sensors, and/or transducers. For example, bioelectrodes and Doppler transducers may be included.

In certain aspects, over 100 structural features of the CBFV waveform will be extracted from the raw CBFV signal collected by the TCD system. In certain approaches, these structural feature algorithms are performed by processing circuitry. The systems and methods described herein further develop and apply these techniques specifically for non-invasive ICP assessment from TCD-based CBFV and/or ECG waveforms for the detection of IH.

FIG. 2 shows a block diagram of the structural feature algorithm. There is a three step process after acquiring raw data: Structural feature extraction, Classification, and Results/diagnosis. The inputs to the system are variable based on the number of vessels; however at least one intracranial vessel is required. A groundtruth (reference data) for the classification is also determined by the neurological condition (mild TBI, severe TBI, stroke, etc.).

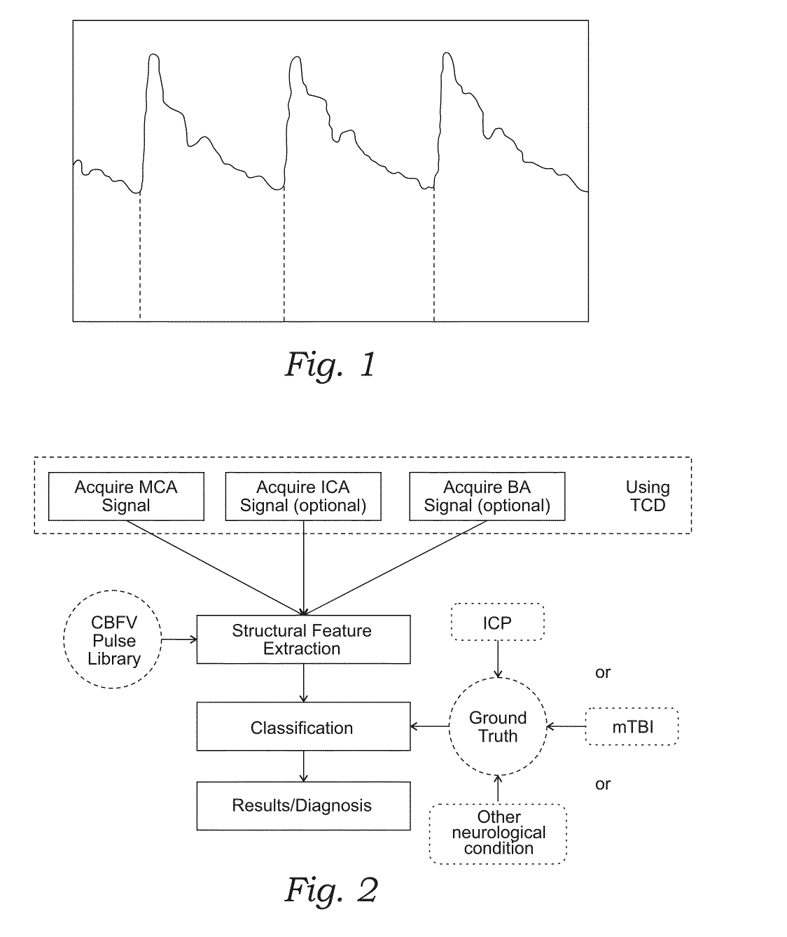

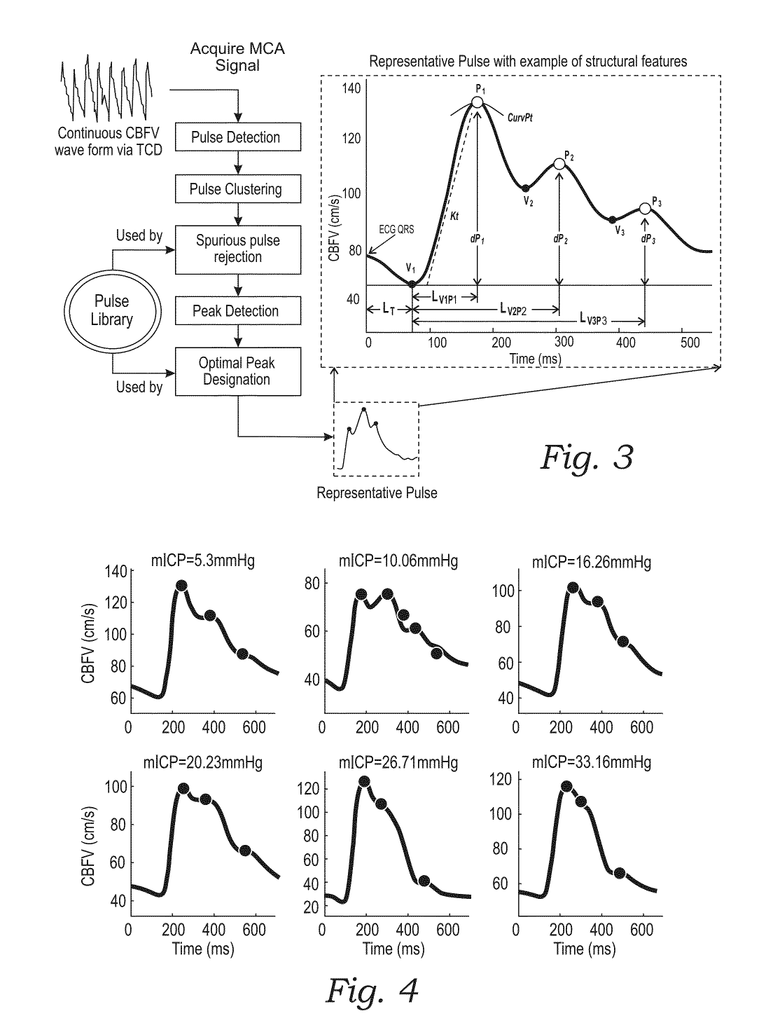

First, individual CBFV pulses from a continuous CBFV segment are extracted in association with a simultaneously recorded ECG segment. FIG. 3 is a block diagram of the structural feature extraction process showing a continuous CBFV input waveform that is transformed into one representative output CBFV pulse with three sub-peaks. The inset to the right shows a schematic representative pulse from a CBFV waveform with the six landmarks (three peaks and three valley points). The maximum velocity envelope shown in FIG. 1 is the input into the block diagram. The identification of the six landmarks is essential for the structural feature extraction.

In certain approaches, the series of individual CBFV pulses is grouped into groups based on correlation coefficient. In certain approaches, the groups of pulses are identified through principal component analysis, correspondence analysis, matrix decomposition, spectrum analysis, independent component analysis, or other waveform signal processing methods. The representative pulse of the group is the average of the largest sub-group, which is identified by the number of pulses within the cluster or group. The representative pulse may be identified through an average of the pulses for the largest sub-group. After constructing the representative pulse, the pulse is validated against a set of previously validated CBFV pulses. The CBFV pulse library may include data sets and representative pulses from many patients/subjects. In certain embodiments, the pulse library includes at least 100 CBFV pulses. In certain embodiments, the pulse library includes at least 10000 and even more CBFV pulses.

The representative pulse is then used for further quantification and diagnosis. In certain embodiments, three subpeaks of the representative pulse are designated among several peak candidates. The insert in FIG. 3 illustrates a typical representative pulse with six landmarks, {P1, P2, P3, V1, V2, V3}, which include three subpeaks and three subtroughs. In certain embodiments, peak locations may be found at using the concave portions of the pulse curve according to four possible definitions in the embodiment shown. The first definition treats the intersection of a concave to a convex region as a peak if the first derivative of the concave portion is greater than zero, otherwise the intersection of a convex region to a concave region is the peak. The second definition is based on the curvature of the signal such that the peak is the location with maximal absolute curvature within each concave region, the third and the fourth definitions both involve a straight line linking the two end points of a concave region. According to the third and the fourth definitions, a peak can be found at the position where the perpendicular distance or the vertical distance from the CBFV to this line is maximal, respectively. Typically, a peak corresponds to the intersection of a convex to a concave region on a rising edge of CBFV pulse or to the intersection of a concave to a convex region on the descending edge of the pulse. This detection process at produces a pool of N peak candidates (a1, a2, . . . , aN). Additionally or alternatively, detection and assignment of peaks may be assigned using a regression analysis, such as spectral regression analysis or multi-linear regression.

In certain embodiments, the structural features (i.e., subpeaks, subtroughs, landmarks) are further characterized through metrics, which are used to identify the ICP status and other neurological conditions or neurological indicators (cerebrovascular reactivity, autoregulation, and neurovascular coupling). In certain approaches, a total greater than 100 structural metrics can be extracted from the representative pulse in association with subpeaks and other structural features. These metrics may include latency, amplitude, curvature, slope, and ratios between subpeaks. In certain embodiments between approximately 1 and approximately 10 metrics are extracted. In certain approaches, at least 10 metrics are extracted. In certain approaches, between approximately 10 and approximately 50 metrics are extracted. In certain approaches at least 50 metrics are extracted. In certain approaches, between approximately 50 and approximately 100 metrics are extracted. In certain approaches, at least 100 metrics are extracted. In certain approaches, greater than 100 structural metrics are extracted.

Typical TCD-based CBFV waveforms are predominantly triphasic, which was previously unknown. Plots in FIG. 4 illustrate typical CBFV waveforms associated with various mean ICP values (mICP, 5-33 mmHg): Top row (normal) and bottom row (hypertensive). CBFV representative waveforms associated with low mean ICP values tend to have more distinct subpeaks than those associated with high mean ICP pulses do. This is one of the main advantages of this framework compared to other as our approach places special emphasis on the subpeaks of the waveform. The difference between the second and third subpeak amplitudes is greater in CBFV representative waveforms associated with high mean ICP pulses than it is in those associated with normal mean ICP pulses. In certain approaches, the subpeak size and/or difference between subpeak amplitudes is used to characterize the ICP as normal or IH.

The method extracts various structural features from TCD-based CBFV waveforms. In certain approaches, this method is performed by processing circuitry. Then, the next step is to learn the association rule (or function) between those CBFV structural features and corresponding labels (e.g., +1 for hypertensive samples and −1 for normal samples). It can be simply expressed as

f(X_{n×128})→Y_{n×1} (1)

where X is an n×100 matrix of structural features, Y an n×1 vector of corresponding labels, n is the number of samples, and f is the association function or classifier to be learned or trained. In certain embodiments, the quality of the trained classifier is measured by its predictive accuracy. In other words, a good classifier is the one that can assign new features, which are unseen during training, into proper classes.

In certain approaches, the learning algorithm includes a graph-based semisupervised learning classification technique, called Spectral Regression. This approach combines the ordinary regression technique with spectral graph analysis and can be used as a clustering and dimensionality reduction technique. In contrast to many conventional graph-based algorithms, which are transductive in nature, the Spectral Regression technique gives a natural out-of-sample extension both in the linear and kernel cases.

The first step of Spectral Regression is to compute a set of responses yᵢ for individual samples xᵢ by applying spectral techniques to a graph matrix. Once those responses are obtained, the ordinary ridge regression technique finds the regression function. The algorithmic procedure of Spectral Regression can be summarized as follows.

1) Adjacency graph construction: Let G denote a graph with n nodes, where the ith node represents the ith sample, xᵢ. Construct the graph G by the following three steps:

a) Connect nodes i and j if they are among k nearest neighbors of each other.

b) Connect nodes i and j if they belong to the same class.

c) Remove the connection between i and j if they belong to different classes.

2) Weight matrix construction: Let W denote a sparse n×n matrix whose element W_{i,j} can be assigned as follows:

$${W}_{i , j} = \{ \begin{matrix}0 , & if nodes i and j are not connected \\ 1 / {l}^{q} , & if {x}_{i} and {x}_{j} belong to the same class \\ s ( i , j ) , & otherwise\end{matrix}$$

otherwise where I^{q} is the number of samples that belong to the qth class and s(i, j) a similarity function between xᵢ and xⱼ. Our choice of this similarity function was the heat kernel, i.e.,

$$s ( i , j ) = {e}^{- \frac{{‖ {x}_{i} - {x}_{j} ‖}^{2}}{2 {σ}^{2}}} . \qquad ( 2 )$$

3) Eigen decomposition: Find the largest eigenvectors of an eigen problem below

Wy=λDy (3)

where D is a diagonal matrix whose element D_{i,i} equals the sum of the ith column of W.

4) Regularized least squares: Solve a regularized least squares problem for the pth largest eigenvector y^{p} as follows:

$${a}^{p} = {argmin}_{a} [ {\sum }_{i = 1}^{l} {( {x}_{i}^{T} a - {y}_{i}^{p} )}^{2} + {\sum }_{i = l + 1}^{n} {( γ {x}_{i}^{T} a - {y}_{i}^{p} )}^{2} + α {‖ a ‖}^{2} ] \qquad ( 4 )$$

where a is a regression coefficient vector, I the number of labeled samples, γ a parameter to adjust the weights of unlabeled samples, and α a regularization parameter. It is important to note that xᵢ is a sample vector while yᵢ a scalar response. By setting γ=1, the closed-form solution of a^{p} can be expressed as

a^{p}=(XX^{T}+αI)⁻¹Xy^{p}. (5)

One of many merits of Spectral Regression is that it provides a uniform learning approach. When samples are all labeled, Spectral Regression is essentially identical to regularized discriminant analysis. In this case, the sparse matrix W becomes block-diagonal and the response y in (3) is equal to

$${y}^{p} = {[ \underbrace{0 , … , 0 ,}_{{\sum }_{i = 1}^{p - 1} {l}^{i}} \underbrace{1 , … , 1 ,}_{{l}^{p}} \underbrace{0 , … , 0}_{{\sum }_{i = p + 1}^{c} {l}^{i}} ]}^{T} \qquad ( 6 )$$

where P is the number of samples that belong to the pth class and c the total number of classes. On the other hand, when samples are all unlabeled, Spectral Regression becomes a spectral clustering technique with a natural out-of-sample extension capability, whose objective function is

$$min {\sum }_{i , j} {‖ {y}_{i} - {y}_{j} ‖}^{2} {W}_{i , j} . \qquad ( 7 )$$

Equation (7) indicates that the responses, yᵢ and yⱼ, should be close to each other when the ith and jth samples are similar. The eigenvectors of the problem in (3) yield the optimal solution of the problem in (7). In the case of semisupervised learning, the responses, yᵢ and yⱼ, as the solution of the eigen problem in (3) can be as close as possible when the ith and jth samples belong to the same class. Such a property is essential for semisupervised learning since the same labeled samples are expected to have the same or similar responses.

Another important merit of Spectral Regression is that it can be easily extended into a nonlinear discriminant analysis by projecting all samples into the reproducing kernel Hilbert space. Then, we can perform Spectral Regression in the high dimensional feature space and it is referred to as spectral regression kernel discriminant analysis (SRKDA). In this case, the closed-form solution of a^{p} in (5) becomes

a^{p}=(K+αI)⁻¹y^{p} (8)

where K is an n×n matrix, whose element K_{i,j} is K(xᵢ, xⱼ) and K(•,•) is the kernel function. In certain approaches, a Gaussian kernel is selected and used. SRKDA was utilized in certain clinical and experimental approaches, as described in further detail below.

There are two important parameters to be optimized in the SRKDA algorithm: standard deviation of the heat kernel σ in (2) and that of the nonlinear (i.e., Gaussian) kernel function, K(•,•). The standard deviation σ of the heat kernel is estimated as follows:

$$\hat{σ} = \sqrt{\frac{1}{n - 1} {\sum }_{i = 1}^{n} {( {x}_{i} - \frac{1}{n} {\sum }_{j = 1}^{n} {x}_{j} )}^{2}} \qquad ( 9 )$$

where n is the total number of training samples. In certain approaches, the parameter σ can be optimized by running a separate cross-validation within a given training dataset. However, there is a risk of overtuning σ to a given training dataset and compromising the generalizability of the model. In contrast, the estimate of σ in (9) is easy to obtain and its value is similar to what could have been obtained by taking the cross validation approach. Therefore, in certain embodiments, the standard deviation of the Gaussian kernel function K(•,•) is estimated as in (9).

### Clinical Examples

In order to validate the systems and methods described herein, a data set comprising ICP, CBFV, and ECG data was collected from 90 patients (ages: 18-92 [median: 47], gender: 47 male/43 female) admitted to neural-ICU and floor units at UCLA Medical Center between Jul. 15, 2008 and Nov. 16, 2011. Among them, 44 patients suffered from TBI, 36 had SAH, and the rest were diagnosed with suspected NPH. Table I summarizes patient's diagnostic and demographic information.

TABLE I
SUMMARY OF PATIENT INFORMATION

Gender

| Diagnosis | Age | Female | Male |
|---|---|---|---|
| TBI | 45 ± 15 | 18 | 26 |
| aSAH | 62 ± 12 | 21 | 15 |
| NPH | 59 ± 10 | 4 | 6 |

TBI: traumatic brain injury.
aSAH: aneurysmal subarachnoid hemorrhage.
NPH: normal pressure hydrocephalus.

ICP was measured invasively via continuous ICP monitoring for the clinical purpose using either intraventricular catheters for brain injury or intraparenchymal microsensors for NPH patients. Simultaneous cardiovascular monitoring was also performed using the bedside GE monitors. CBFV signals were obtained at the MCAs, which was ipsilateral to the ICP measurement location, while technicians affiliated with the Cerebral Blood Flow (CBF) laboratory at UCLA Department of Neurosurgery conducted daily clinical assessment of patients' cerebral hemodynamics using TCD. The duration of collected signals varies depending on how long the TCD monitoring of the MCA could be done. Typically, the TCD monitoring lasted only 3-5 min since the probe had to be hand-held. This study was approved by Institutional Review Board without involvement of any personal health information.

All signals were archived via a mobile cart equipped with the PowerLab data acquisition system (ADInstruments, Colorado Springs, Colo.), which samples analog signals from the bedside monitor at 400 Hz. Then, the archived signals were stored into the Chart binary file format for further analysis.

ICP range was divided into three groups: normal<(15 mmHg), gray-zone (15-30 mmHg), and IH (>30 mmHg). ICP remaining below 15 mmHg is assumed to be indicative of a normal state. In contrast, a patient's condition is assumed to be at a greater risk when the ICP is beyond 30 mmHg.

ICP and CBFV segments of 3-5 min lengths, which were simultaneously recorded during each session of daily cerebral hemodynamics assessment, were broken down into 1-min segments. Each of these 1-min segments was used to contribute one sample, that is, a set of the CBFV structural features. From 90 patients, 563 samples were obtained over 131 sessions. Those samples were assigned labels by applying the labeling criteria described above on the session level, not the sample level. In other words, if any of samples belonging to a given session meets the IH criterion, all samples of the session are labeled as IH. The rationale behind this labeling scheme is that what caregivers are most concerned about is whether a patient experiences IH at all during a given session. Which of the 1-min segments during the session is associated with IH is typically not of much interest. However, in certain approaches identification of the specific time of the IH occurrence or occurrences is provided. In contrast, a given session is labeled as Normal only when all the samples within the session meet the normal (i.e., <15 mmHg) criterion. Any session that is not labeled as IH or Normal is labeled as Gray-zone. Table II summarizes the results of our labeling scheme. It is important to note that only some of 48 samples from eight IH sessions correspond to ICP above 30 mmHg, while all the samples from 46 Normal sessions correspond to ICP below 15 mmHg.

TABLE II
SUMMARY OF DATA LABELING

| Labels | Samples | Sessions | Patients |
|---|---|---|---|
| IH | 48 | 8 | 8 |
| Normal | 150 | 46 | 34 |
| Gray-zone | 365 | 77 | 48 |
| Total | 563 | 131 | 90 |

With the labeling scheme described above, we performed two separate cross-validation experiments. The purpose of the first cross-validation experiment was to quantify the performance of SRKDA to differentiate IH samples from normal ones. In the first cross-validation experiment, the tenfold cross-validation was performed only over the IH and normal samples, where the gray-zone samples are used just for the training purpose. We use those gray-zone samples in three different ways: Supervised¹, Supervised², and Semisupervised. In the setting of Supervised¹, the gray-zone samples are labeled as IH or normal based on the conventional IH threshold of 20 mmHg and used as “labeled” samples for the training purpose. In the setting of Supervised², they are considered as “noisy” samples and discarded completely. Finally, in the setting of Semisupervised, they are used just as “unlabeled” samples for the training purpose. We also considered the PI-based IH detection as our baseline classifier and compared its performance against our proposed methods.

The purpose of the second cross-validation experiment was to examine whether SRKDA can cluster the gray-zone samples according to their corresponding ICP values. In this experiment, the tenfold cross-validation is performed only over the grayzone samples in a semisupervised learning fashion, where all IH and normal samples are used just for the training purpose. While the label of hypertensive samples is +1 and that of normal ones is −1, the direct output of SRKDA is a continuous-scale estimate of the label. We were mainly interested in whether these continuous-scale estimates of the gray-zone samples are strongly correlated with their corresponding ICP values.

It is important to note that all cross-validations in our study were conducted in the leave-patients-out manner. If some samples from one patient are used for the training purpose, none of samples from the same patient can be used for the testing purpose. The performance of IH detection is calculated on the session level not on the sample level. As described above, it is of much interest to know whether individual sessions are associated with IH. Since the direct outputs of SRKDA are continuous-scale label estimates of individual samples, we aggregated all samples that belong to a given session and chose the maximum valued estimate of the label as the session's label.

The following sections describe two distinct performance measures, i.e., area under the curve (AUC) and decision curve analysis, which we used in our study.

1) Area Under the Curve: The predictive accuracy is measured by the area under the receiver operating characteristic (ROC) curve. The area under the ROC curve can be thought of as the probability that the rank of a randomly chosen positive sample is higher than that of a randomly chosen negative one. By plotting the AUC of the semisupervised SRKDA against the number of close neighbors, k, we examined the effect of k on the performance of the semisupervised classifier.

2) Decision Curve Analysis: AUC as a predictive accuracy measure does not weigh clinical consequences of false-positive and false-negative results. In other words, it cannot tell us whether using a given diagnostic method is clinically useful at all. For example, when missing a diagnosis is more harmful than treating a disease unnecessarily, a diagnostic method A with a higher sensitivity would be a better clinical choice than another diagnostic method B with a higher specificity but a lower sensitivity although the AUC of the method A can be slightly smaller than that of the method B. In order to evaluate and compare different diagnostic methods by incorporating clinical consequences, we used decision curve analysis. The decision curve analysis derives the net benefit (i.e., clinical advantage) of a given diagnostic method across a range of the disease probability threshold pt. It assumes that the disease probability threshold pₜ, at which a patient would opt for treatment (invasive ICP monitoring in our case), reflects the patient's weighing on necessary (true positive) and unnecessary (false positive) treatments. However, there is no apparent reason to focus solely on those individuals who opt for treatment when calculating the net benefit. Recently, a modified net benefit for all individuals with and without treatment. This overall net benefit can be expressed as:

$$net benefit = \frac{no . of true positives + no . of true negatives}{no . of total samples} - \frac{no . of false positives}{no . of total samples} ( \frac{{p}_{t}}{1 - {p}_{t}} ) - \frac{no . of false negatives}{no . of total samples} ( \frac{1 - {p}_{t}}{{p}_{t}} ) . \qquad ( 10 )$$

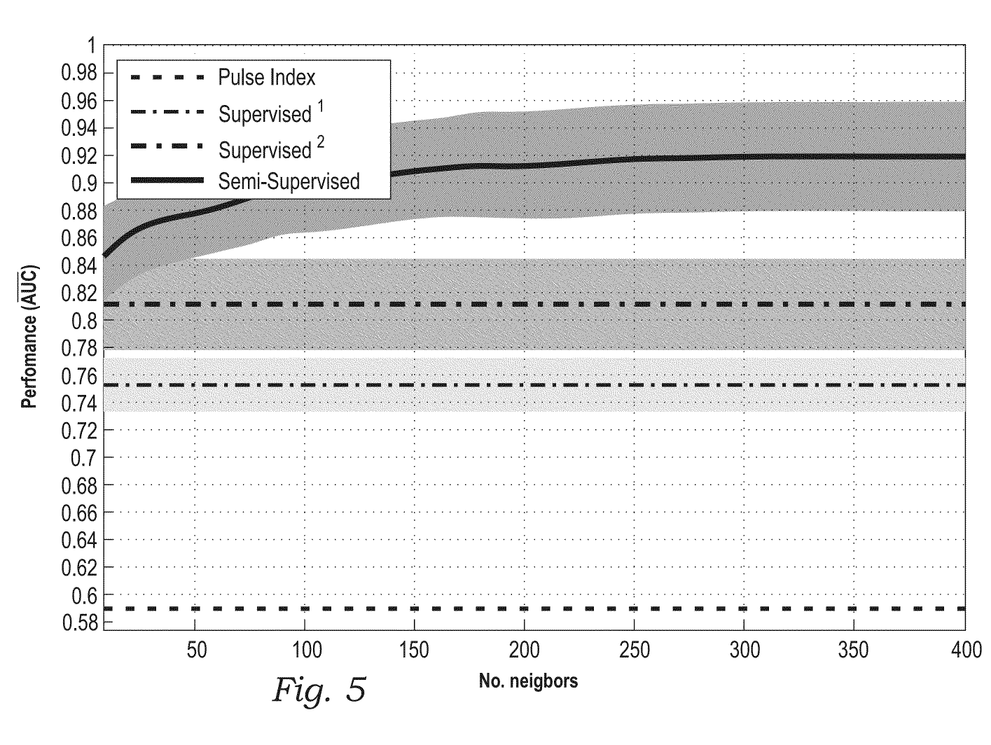

FIG. 5 compares the AUC of four IH detection methods in the first cross-validation experiment, where the dashed green line is for the PI-based IH detection method (baseline method), the thin dashed-dotted blue line for the Supervised¹ IH detection method, the thick dashed-dotted light-blue line for the Supervised² IH detection method, and the solid red line for the Semisupervised^{k} IH detection method. Since only the Semisupervised^{k} IH detection method has to do with the number of neighbors to explore, k, the AUC of all other methods remained constant across the entire range of k. Each line and gray area represent the mean AUC and one standard deviation variation over multiple (=100) tenfold cross-validations. There are several interesting aspects to point out in FIG. 5. First, all of our proposed IH detection methods are substantially better than the PI-based IH detection method. Second, the Supervised¹ IH detection method is slightly worse than the Supervised² IH detection method. It indicates that utilizing the gray-zone samples as labeled data based on the 20 mmHg threshold actually worsens the predictive accuracy of the SRKDA classifier. Third, the AUC of the Semisupervised^{k} IH detection method tends to increase as k increases.

TABLE III
SUMMARY OF OVERALL NET BENEFIT GAINS

| Method | PI | Supervised¹ | Supervised² | Semi⁵⁰ | Semi²⁰⁰ |
|---|---|---|---|---|---|
| Gain | 0.04 | 0.11 | 0.10 | 0.16 | 0.19 |

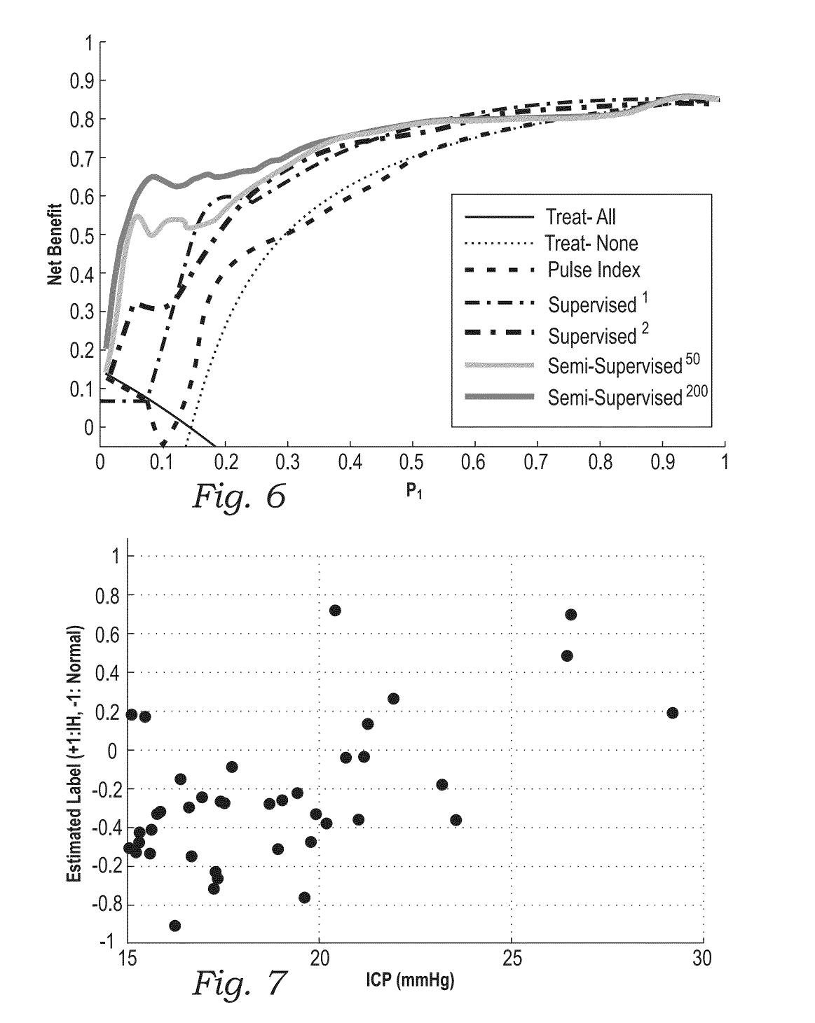

FIG. 6 illustrates the decision curves (net benefit versus probability threshold, pₜ) of the IH detection methods in the first cross-validation experiment. The net benefit of the PI-based IH detection method (dashed green line) is slightly better than that of two extreme approaches (i.e., Treat-All and Treat-None) only over a very narrow range of pt from 0.14 to 0.27. In contrast, the net benefit of our proposed methods based on the structural features is significantly better than that of two extreme approaches over a wide range of pₜ.

FIG. 5 also reveals the superior performance of the semisupervised IH detection methods over the supervised methods in a qualitative sense. However, it may not be trivial to make a quantitative performance comparison since the decision curves in FIG. 6 cross over one another. Table III summarizes each IH detection method's net benefit gain as the averaged difference between the net benefit of each IH detection method and that of two extreme approaches across the entire range of pt. The net benefit gain attempts to measure the degree of true net benefit that can be achieved by using a specific IH detection method over two extreme approaches (i.e., Treat-All and Treat-None). The net benefit gains listed in Table III clearly demonstrate that the semisupervised IH detection methods are significantly better than the other methods and the PI-based IH detection method is not any better than the Treat-All and Treat-None approaches.

FIG. 7 visualizes the results of the second cross-validation experiment where the continuous-scale label estimates of the gray-zone samples are on y-axis and the corresponding ICP values on x-axis. The continuous-scale label estimates tend to increase as the corresponding ICP values increase and the correlation coefficient between them was 0.55 with 2e-4 p-value.

The regularization parameter α in (4) is to prevent overfitting of the least square solution a^{p} by penalizing its complexity, i.e., ∥a∥². In certain approaches, this parameter can be optimized by running a separate cross-validation within a training dataset. Instead, by testing SRKDA on preliminary datasets, we learned that the regularization parameter α does not affect the performance of SRKDA significantly as long as its value remains small (<0.01). Accordingly, in certain approaches, such as the clinical dataset and analysis described herein, α is set at 0.01.

In certain approaches, such as those used for analysis of the clinical data described herein, feature selection methods are not used, although the correlation between some structural features is likely. Accordingly, in certain approaches, feature selection methods utilizing correlations between features are implemented. Nonlinear kernel-based classification methods such as SRKDA are efficient in classifying high-dimensional data so that feature selection or feature weighting is not necessary for the purpose of classification. For the present data, feature selection techniques provided no noticeable performance improvement for the IH detection method. However, it should be noted that the time delay between the ECG-QRS and the first trough of CBFV as shown in FIG. 3 was the single most important feature for accurate IH detection. By simply excluding this feature from our simulation study, the performance of IH detection deteriorated by ≈10% on average. There was no other subset of features that affected the performance of IH detection to that extent.

Our cross-validation results in FIGS. 5 and 6 clearly indicate that CBFV PI does not reflect elevated ICP very well as compared to using the complete set of pulse structural metrics. The variation in the reported PI-ICP correlation behavior could be attributed to the fact that CBFV PI is influenced by many other factors including arterial blood pressure and age. In addition, there are three very different patient populations in this study, which further confounds the PI-ICP relationship. The superior performance of our approach may indicate that the SRKDA model may be able to implicitly select the discriminative features from the provided set of structural metrics that are less confounded by the factors not related to ICP status.

The performance (i.e., predictive accuracy) of the semisupervised IH detection method improves as the number of close neighbors (or samples) k increases as shown in FIG. 5. This finding can be accounted for by pointing out the fact that the weight matrix W becomes denser with a large k and the intrinsic data structure among unlabeled and labeled samples can be explored more extensively to improve the predictive power of SRKDA. The decision curve analysis results in FIG. 6 and Table III also support the idea that the semisupervised IH detection method can perform better with a large k.

The performance of the proposed IH detection method on a sample level was significantly lower than that on a session level. One possible explanation is that CBFV may respond to ICP elevation in a delayed fashion due to CBF autoregulation. When acute ICP elevation occurs, an intrinsic physiological delay is inevitable to see CBFV pulse structural changes. That delay is usually 10-20 s for intact autoregulation. Therefore, in certain approaches, IH detection is used on a session level.

The ROC curve analysis is solely focused on the accuracy of a given prediction model, while the decision curve analysis concentrates on the utility of the model. As a result, the optimal operating point based on the latter is quite different from that based on the former. Typically, the optimal operating point based on an ROC curve is the one where the Youden index (i.e., sensitivity+specificity−1) is maximized. This optimal operating point and corresponding threshold will be referred to as the optimal accuracy operating point and optimal accuracy threshold pₐ. However, the net benefit of a prediction model with the optimal accuracy threshold pₐ drops below that of two extreme approaches as soon as pₜ departs from the optimal accuracy threshold. This optimal operating point and corresponding threshold will be referred to as the optimal net benefit operating point and optimal net benefit threshold. The optimal net benefit operating point on the ROC curve can be determined as the point whose slope is equal to [(1−π)/π][pₜ/(1−pₜ)], where π is the portion of all positive samples. This optimal net benefit operating point is “optimal” in a sense that it maximizes the net benefit at a specific value of pₜ.

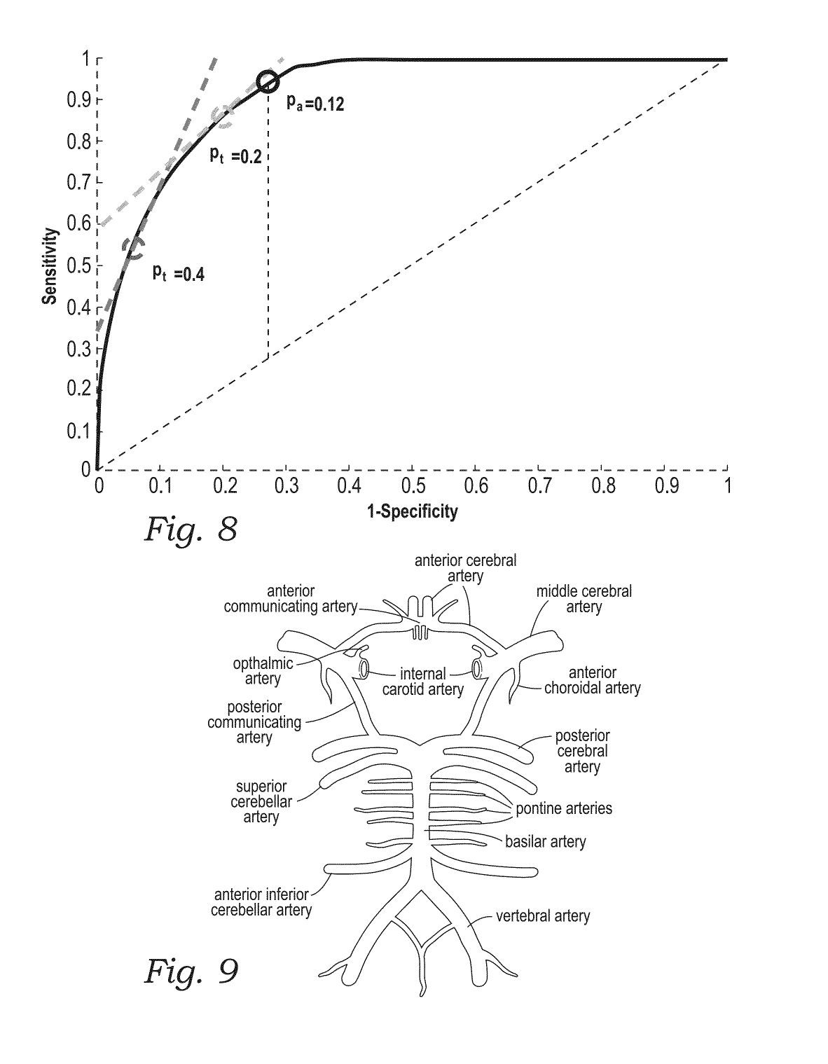

FIG. 8 shows three different operating points on the ROC curve of the semisupervised²⁰⁰ IH detection method, where the red dot is for the optimal accuracy operating point with pₐ=0.12, the green dot is for the optimal net benefit operating point for pₜ=0.2, and the blue dot is for the optimal net benefit operating point for pₜ=0.4. The semisupervised²⁰⁰ IH detection method with pₐ=0.12 may yield the optimal accuracy performance. However, it can yield a better net benefit than the Treat-All or Treat-None approach only when pₜ is close to 0.12 and it is virtually useless when a high value of pₜ is selected. FIG. 8 well illustrates why a highly sensitive prediction model is preferred with a small value of pₜ while a highly specific prediction model is preferred with a large value of pₜ.

An IH diagnostic tool as described herein can be used in a diverse set of clinical applications where an appropriate pₜ may be different. As such, it is very useful to conduct the decision curve analysis to help select different models and their operating points to fit the intended usage of obtaining an IH diagnosis.

However, it remains interesting to investigate whether an SRKDA model trained using data from brain injury and hydrocephalus patients can extrapolate well to the IIH patient population although our results have indicated that using a set of CBFV pulse structural metrics is more promising than using a single metrics such as PI with regard to handling data from a heterogeneous patient population.

The ICP level of 20 mmHg is a conventional threshold to define IH instances. However, it is somewhat arbitrary and tends to cause many false positive alarms. In certain approaches, the systems and methods described herein divide the ICP range into three groups: normal (<15 mmHg), gray-zone (15-30 mmHg), and IH (>30 mmHg). By adopting the SRKDA algorithm, we have demonstrated that the semisupervised learning approach, where gray-zone samples are treated as unlabeled data, is more suitable for IH detection than the traditional supervised learning approach.

It should be understood that the above steps, such as those described and those shown in the flow diagrams, may be executed or performed in any order or sequence not limited to the order and sequence shown and described in the figure. In certain approaches, steps may be excluded. In certain approaches, steps may be added or combined. Additionally or alternatively, some of the above steps may be executed or performed substantially simultaneously where appropriate or in parallel to reduce latency and processing times.

The methodologies disclosed herein are preferably enabled by using an Ultrasonic Transducer Positioning mechanism with a Transcranial Doppler (TCD) system that is designed to detect potential brain trauma by monitoring cerebral blood flow. This is accomplished by positioning ultrasonic transducers on either side of the patient's head and optimally positioning the transducers to maximize the ultrasonic Doppler flow signal.

In use, an Ultrasonic Transducer Positioning mechanism (UTPM) is placed adjacent to the temporal region on both sides of the patient's head. The intersection of the patient's head and upper ear lobe provides a reference landmark for placement of the mechanism enclosure. Enclosure position relative to the head is desirably maintained via attachment to a separate headgear appliance, though a handheld probe as shown may be used.

The Ultrasonic Transducer Positioning mechanism seeks the optimal location on the patient's head to provide the best Doppler flow signal via minimum bone attenuation and zero degree angle of insonation to the cerebral artery. Namely, the mechanism positions the transducer under direction of a processing unit which strives for signal maximization via XYZ+XY tilt commands to the mechanism drive circuitry. Preferably, the mechanism is capable of autonomous scan and positioning.

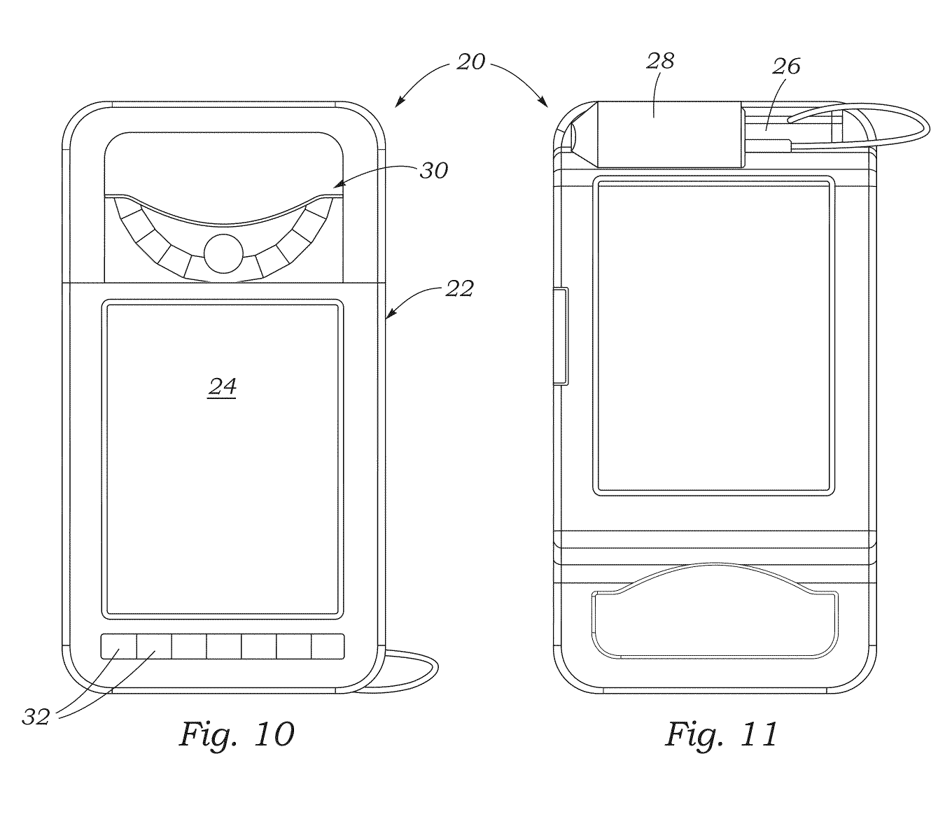

FIGS. 10 and 11 are front and rear views of a portable transcranial Doppler device 20 for use in collecting CBFV raw data as described herein. The device 20 includes a main body 22 having a size and shape much like a conventional smart phone, with a display screen 24 which may be a touch-sensitive LCD. An ultrasound probe 26 stores within a holster 28 on the back of the device and may be secured magnetically. Various controls may be provided in an upper panel 30 or as buttons 32 below the screen 24. The portable device will work with either hand and the display screen 24 may adjust to the given direction. The technician removes the ultrasound probe 26 from the holster 28 and applies it to an area on the head of the patient, typically around one of the temples. Measurements of CBFV raw data are then taken for a period of time and recorded. The same process scan be repeated at different locations, and is entirely non-invasive. Preferably, an ultrasonic coupling gel such as typically used for fetal ultrasound probes is used to enhance comfort to the patient and improve transmission of the ultrasonic waves through the epidermis and dermis.

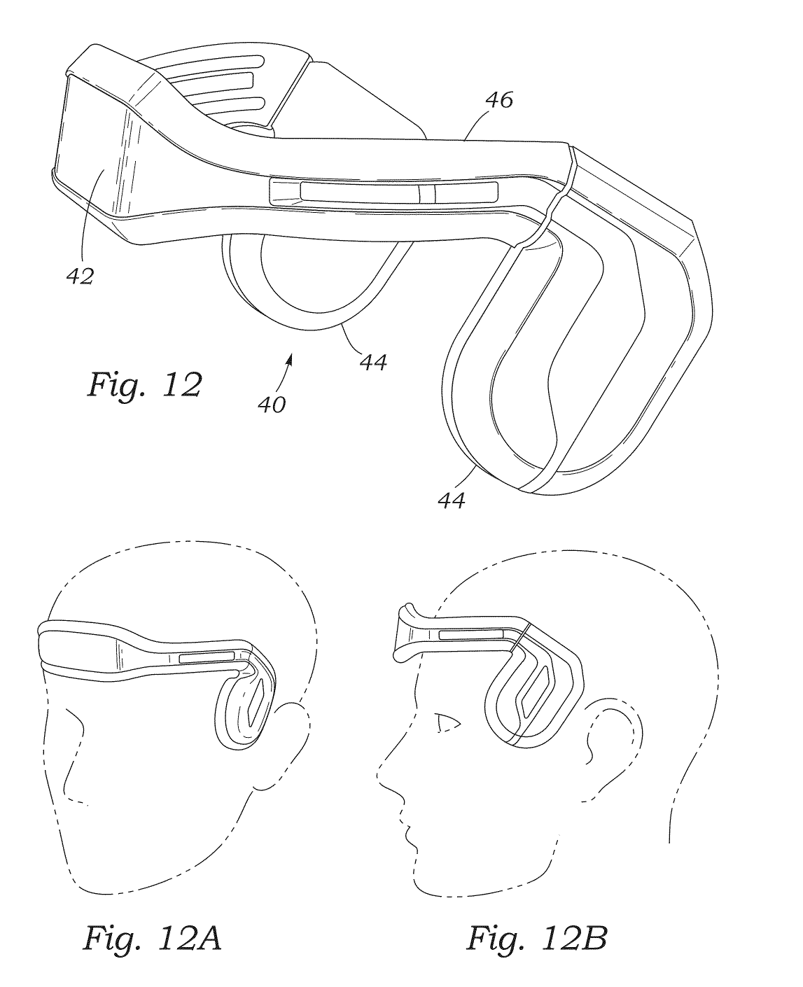

FIG. 12 shows an automated TCD headset 40 having a display screen 42 on the front thereof. More particularly, the headset 40 includes dual ultrasound probes 44 on the sides and a headband 46 that extends around the front so as to connect the two probes. As seen in FIGS. 12A and 12B the TCD headset 40 fits over the cranium of a patient with the probes 44 located at either temple. The probes 44 include TCD scanners therein that can auto locate the middle cerebral artery (MCA). Desirably, the headband 46 is elastic in nature and enables the headset 40 to fit snugly over the front of the head of a variety of different head sizes so that the inner face of the probes 44 akes good contact with the temples. Again, a lubricating gel is preferably used to improve acoustic transmission.

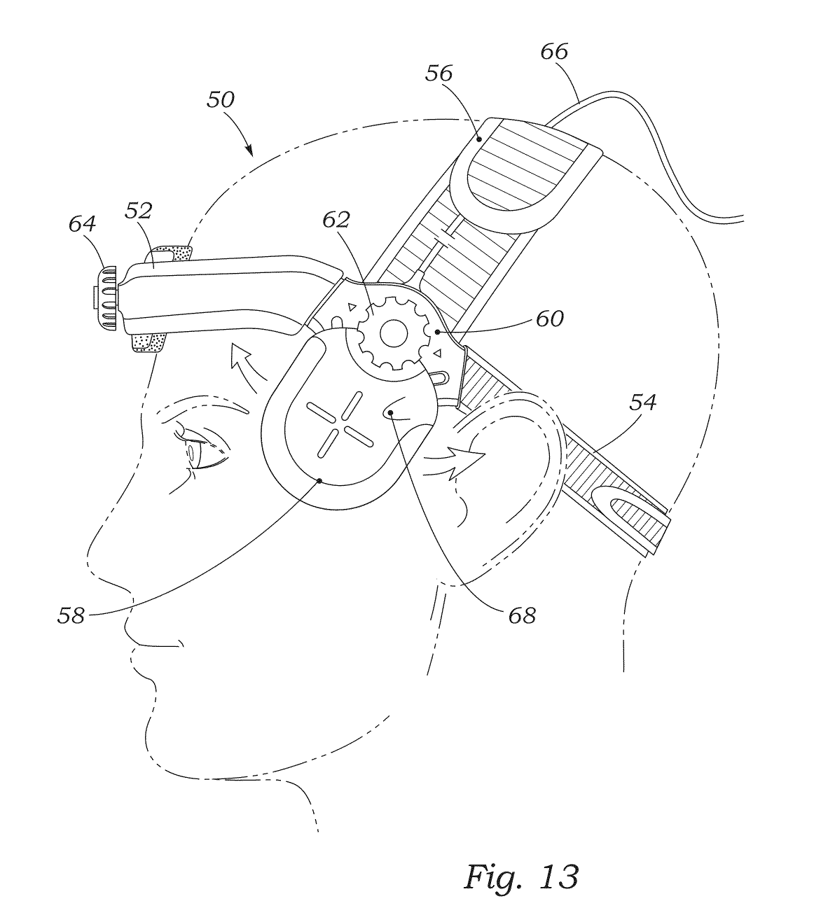

FIG. 13 is a side view of another exemplary TCD headset 50 worn by a patient and having a forehead strap 52, a rear strap 54, and a cranial strap 56. The straps 52, 54, 56 help secure the headset 50 on the head, and in particular ensure good contact of a pair of reciprocating TCD scanners 58 with either temple. The TCD scanners 58 mount for reciprocal forward and backward rotation, as indicated by the movement arrows, to a junction member 60 at the intersection of the three straps 52, 54, 56. In one embodiment, the TCD scanners 58 rotate about 60° in each direction about a Z-axis perpendicular to the XY scan plane. Although not shown, a small motor within the junction member 60 enables movement of the scanners 58.

The system of the three straps 52, 54, 56 is extremely effective in holding the headset 50 in place. The cranial strap 56 includes a Velcro break for adjustability, the rear strap 54 is desirably elastic, and a pair of tightening knobs 62 on each junction member 60 and a tightening knob 64 at the middle of the forehead strap 52 enable fine adjustment of the position of the scanners 58 for X-Y calibration. The cranial strap 56 helps limit migration of the headset 50 once secured due to movement of the jaw and associated muscles.

A cable 66 may be attached to the junction members 60 for connection to a control unit such as a tablet computer, or the system may be wireless. Each scanner 58 desirably includes an injection port 68, preferably formed by an indent leading to a channel, for introduction of a lubricating gel to the inside contact surfaces. This helps reduce a messy application of the gel. In a preferred embodiment, the TCD sensor on the inside of each scanner 58 may be displaced in the Z-direction, or toward and away from the temple, to optimize acoustic contact.

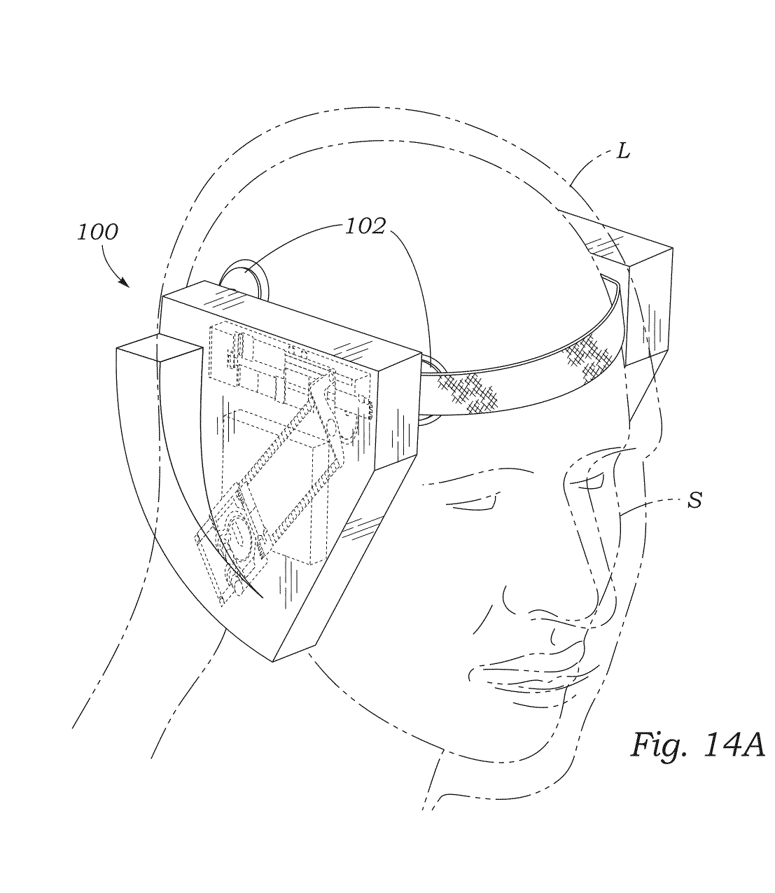

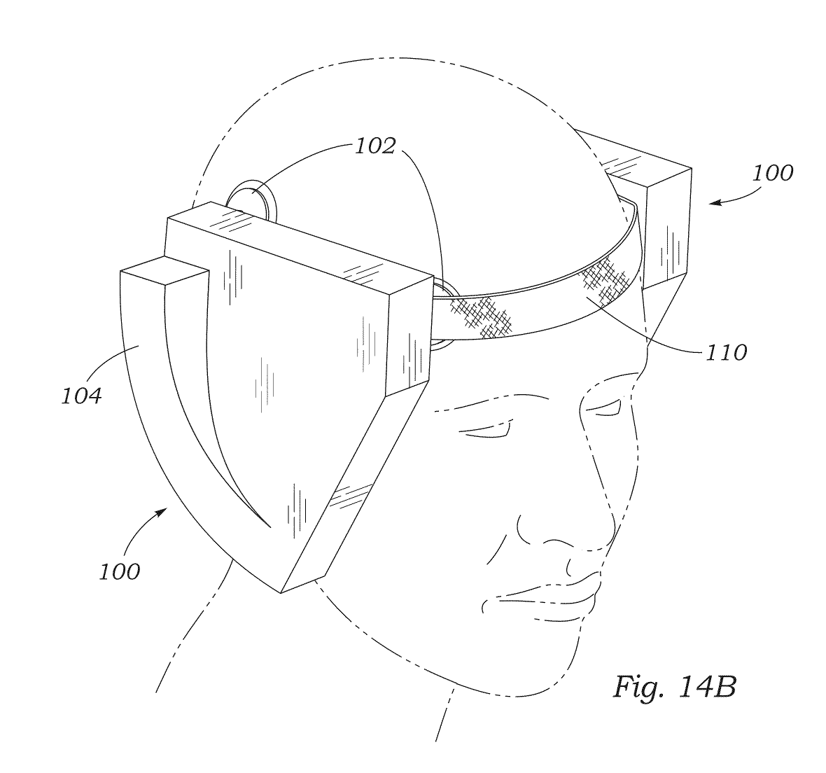

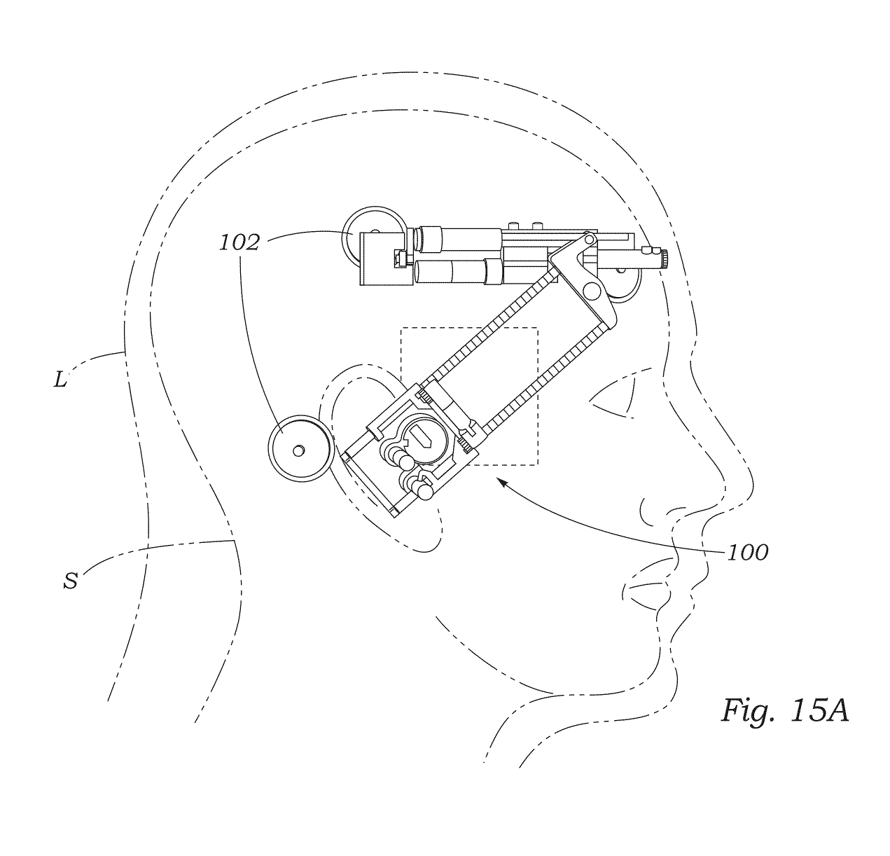

FIG. 14A is a perspective views of an exemplary TCD headset 100 positioned on soft mounting feet 102 on the side of a patient's head. Two sizes of patients' heads, small S and large L, are shown in contour lines to indicate the range of adjustability of the headset 100 for different sizes of patients. An outer housing 104 is shown in phantom to visualize internal components of the headset 100.

FIG. 14B shows the outer housing 104 against a profile of the wearer's head for clarity, and also shows a second headset 100 on the opposite side of the patient's head connected to the first set by straps 110. Preferably, each headset 100 has a plurality of the mounting feet 102 which resemble small suction rings to cushion the sets against the head and also provide some spacing between the head and the outer housing 104. There are desirably three mounting feet 102 on each side. The headsets 100 are anchored by tensioning the straps 110. There may be one forehead strap 110 as shown, or also one around the rear and even one over the cranium, as was described above.

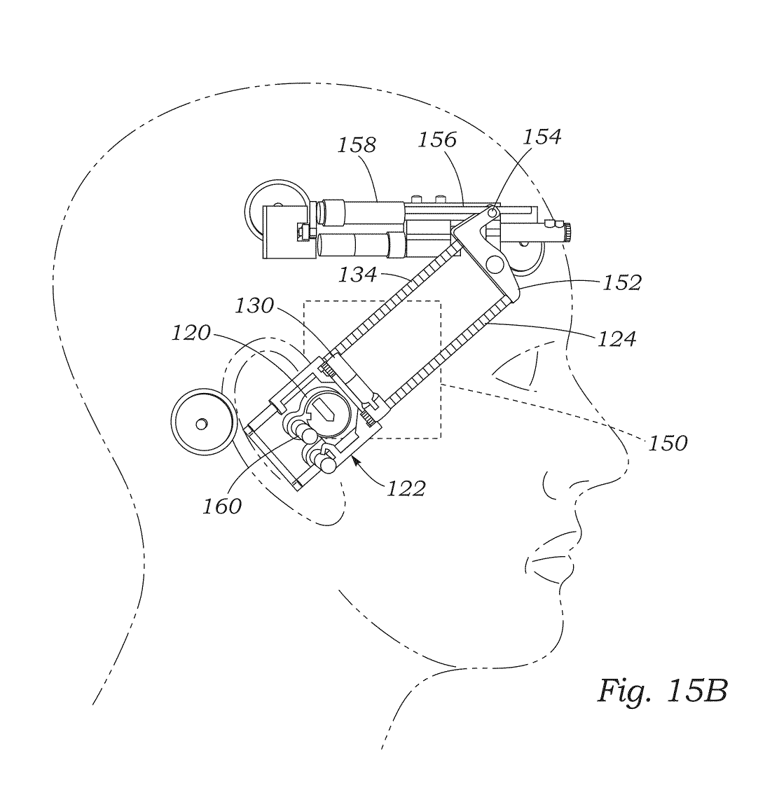

With reference to FIGS. 15A and 15B side elevational views of the TCD headset 100 of FIGS. 14A and 14B are shown with the housing 102 removed. Within the housing, a scanner 120 mounts on a carriage 122 that slides on a pair of diagonal rails 124. The carriage 122 includes a small motor 130 that turns drive gears that mesh with small teeth 134 along both rails 124. The motor 130 may be controlled remotely or by wires, and the carriage 122 thus may be moved diagonally along the rails 124.

The TCD scanner 120 mounted on the carriage 122 thus may be moved over the temple area of the subject. The headset 100 can desirably scan an area of about 2 sq in as indicated by the dashed square area 150. To cover the entire area 150, the upper ends of the rails 124 pivotally attach to a frame member 152 that translates laterally along a generally horizontal path. More specifically, a pivot point 154 on the frame member 152 connects to a translating rod 156 that may be moved by a cylinder 158 in a piston/cylinder relationship. Alternatively, the cylinder 158 may contain a small motor which engages the end of the rod 156 opposite the pivot point 154 and translates it laterally. There are several ways to accomplish this movement, and each is controlled along with movement of the carriage 122 for coordinated two-dimensional movement of the scanner 140 in the XY plane over the target area 150.

In addition, the robotic arm encompassing the scanner 140 mounted for movement on the carriage 122 has a Z-axis displacement device preferably actuated by a stepper motor 160. The robotic arm is further equipped with a pressure sensor (not shown) that maintains sufficient pressure of the scanner 140 against the skin for consistent signal quality. This constant pressure will help address some of the variability issues associated patient movement and TCD.

In terms of preferred mechanisms, translational motion along the XYZ axis+XY Tilt will be accomplished through use of stepper motors driven by a local Motion Control Unit (MCU). Servo feedback will be provided to assure that the commanded number of steps has been accomplished. The servo feedback signal will take the form of a reverse EMF or encoder signal provided to the MCU.

Command Set:

XYZ axis+XY Tilt movement will be controlled via a TPU processor. A command for movement along any axis will be in the form of a signed integer number indicating the number of step increments to be moved along each axis. There are preferably Tilt/Swivel movement controls as well.

A unit that can adjust to several head sizes is important for wide-spread adoption. If the head mount does not fit correctly the TCD probes cannot acquire the optimal signal. The disclosed design addresses this concern separating the “anchoring” of the headset and the robotic mechanism. This allows the user to fit the headset on any sized head with no impact on the ultrasound mechanism to reach the signal.

Each of the headset embodiments is capable of being cleaned of all ultrasonic coupling gel following use. Preferably, wipes or other such devices are provided to protect the mechanism from accumulation of foreign matter within the mechanism. Materials selected must withstand cleaning with water, isopropyl alcohol, and other cleaning agents routinely used in the doctor's office and clinical setting. In a preferred form the headsets shall not weigh more than 10 ounces.

The foregoing is merely illustrative of the principles of the disclosure, and the systems, devices, and methods can be practiced by other than the described embodiments, which are presented for purposes of illustration and not of limitation. It is to be understood that the systems, devices, and methods disclosed herein, while shown for non-invasive diagnosis of IH using TCD, may be applied to systems, devices, and methods to be used in other procedures, including other diagnostic or therapeutic procedures or procedures outside of physiological applications including: diagnosis of cerebral malaria, mild/moderate traumatic brain injury, and others.

In certain embodiments, the systems and methods described could be used for the diagnosis of mild and moderate TBI where there is no increase in ICP. The underlying physiology is different; however, the core analysis is the same. The cerebral hemodynamic changes following a mild TBI are well documented by several studies. The physiologic origin of these changes range from regional blood flow variations owing to increased metabolic demand in certain regions of the brain to variations in CBF due to disruptions in the cerebral vasculature or the brain itself (such as decreased compliance due to high intracranial pressure—ICP).

For instance a study by Jaffres et al. (Jaffres, P., et al., Transcranial Doppler to detect on admission patients at risk for neurological deterioration following mild and moderate brain trauma. Intensive Care Med, 2005. 31(6): p. 785-90) investigated the use of Pulsatility Index (PI) of the CBFV in mild and moderate TBI in the emergency room for prognostic purposes; their results showed that PI alone was able to differentiate patients who had secondary neurological deterioration (SND) from those who did not. A study by Bouzat et al. (Bouzat, P., et al., Transcranial Doppler to screen on admission patients with mild to moderate traumatic brain injury. Neurosurgery, 2011. 68(6): p. 1603-9; discussion 1609-10.) confirmed these results and reported 95% overall accuracy in identifying patients who would develop SND.

Moreover, a number of studies have investigated a possible root cause of the physiological deficit in mild TBI, a decrease in CBF. (see, e.g., Giza, C. and D. A. Hovda, The Neurometabolic Cascade of Concussion. J Athl Train, 2001. 36(3): p. 228-235; and Grindel, S. H., Epidemiology and pathophysiology of minor traumatic brain injury. Curr Sports Med Rep, 2003. 2(1): p. 18-23).

An important study by Maugans et al. (Maugans, T. A., et al., Pediatric sports-related concussion produces cerebral blood flow alterations. Pediatrics, 2012. 129(1): p. 28-37.) using phase-contrast angiography in children with sports-related concussions reports two main results. First, there was a significant decrease in CBF in children aged 11-15 years within 72 hours of the mild TBI. Second, after 14 and 30 days post-injury, only 27% and 64% of patients, respectively, had returned to the normal CBF range despite being asymptomatic after 14 days. Furthermore, a related study by Gall, et al. (Gall, B., W. S. Parkhouse, and D. Goodman, Exercise following a sport induced concussion. Br J Sports Med, 2004. 38(6): p. 773-7.) reported that post-concussed hockey players displayed differential heart rate responses when stressed by exercise, despite the absence of post-concussion symptoms. Both studies demonstrate that despite athletes being asymptomatic there remains a physiological deficit that could be detrimental if further injury or activity were sustained. Finally, in a study by Len et al. (Len, T. K., et al., Cerebrovascular reactivity impairment after sport-induced concussion. Med Sci Sports Exerc, 2011. 43(12): p. 2241-8.) mild TBI was shown to negatively impact cerebrovascular reactivity (CVR) when compared with controls. The results showed that the CVR testing differentiated the concussed and non-concussed athletes. These results echoed those of Gall, et al., which showed that asymptomatic individuals when stressed would exhibit physiologic changes.

One approach to investigate the underlying physiology of mild TBI is to provide a stimulus to exacerbate changes in the cerebrovasculature and use our described framework to more accurately quantify the changes. Stimulus can be provided in a variety of different ways including changes in arterial blood pressure (exercise, leg cuff, pharmaceuticals, etc.), changes in concentrations of carbon-dioxide (CO2) in the arterial blood supply, or local by altering metabolism in specific area of the brain (i.e. flashing lights stimulates the occipital lobe).

In one technique, the cerebrovascular bed is extremely sensitive to changes in arterial blood concentrations of CO₂ (PaCO₂). Increased arterial CO₂ levels (such as from holding one's breath) cause arteriolar vasodilatation resulting in increased velocity in the upstream large cerebral arteries due to increased cerebral blood flow. Conversely, a decreased CO₂ (via hyperventilation) results in decreased CBFV due to arteriolar vasoconstriction causing a reduction in CBF.

Cerebrovascular reactivity (CVR) describes the changes in CBFV due to changes in the PaCO₂. The goal of CVR testing is to assess the vasodilatory or vasoconstrictory capacity of the resistance arterioles of the brain and has been shown to be impaired after a severe TBI, migraine, long-term spaceflight, stroke, and carotid artery stenosis. More recently, CVR has shown potential as marker of physiologic dysfunction in mild TBI by Len et al., infra. In their work, both concussion and control subjects were studied using breath holding and hyperventilation to investigate CVR. Similar to the Gall et al. study, which used exercise as a physiological stress to elucidate changes in concussion patients, Len et al. showed alterations in mean CBFV dynamics from repeated breath holding and hyperventilation. However, the CBFV data was sampled at 1 Hz, removing all morphological information from the analysis. In the present application, the CVR testing utilized by Len et al. is expanded to look at the effect on not just the mean velocity, but the entire shape of the CBFV waveform. The patient is asked to hold his or her breath to raise CO₂ levels and the CBFV monitored. Conversely, the patient is asked to hyperventilate to lower CO₂ levels and the CBFV monitored. Looking at CVR using ONLY mean velocity as in Len, et al. provides an incomplete picture.

While several embodiments have been described that are exemplary of the present system and methods, one skilled in the art will recognize additional embodiments within the spirit and scope of the systems and methods described herein. Modification and variation can be made to the disclosed embodiments without departing from the scope of the disclosure. Those skilled in the art will appreciate that the applications of the embodiments disclosed herein are varied. Accordingly, additions and modifications can be made without departing from the principles of the disclosure. In this regard, it is intended that such changes would still fall within the scope of the disclosure. Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented. Therefore, this disclosure is not limited to particular embodiments, but is intended to cover modifications within the spirit and scope of the disclosure.

1. A non-invasive method for diagnosing a pathological intracranial pressure condition in a patient, comprising the steps of:
non-invasively transmitting and receiving reflections of ultrasound waves to a cranium of a patient using a portable ultrasound transceiver;
processing the reflected ultrasound waves by collecting raw data indicative of cerebral blood flow velocity from at least one blood vessel disposed within a cranial area of the patient;
converting the raw data into structural features using a database of previously-validated cerebral blood flow velocity waveforms;
classifying the structural features using a database of previously identified pathological intracranial pressure conditions; and
recommending a diagnosis based on the step of classifying.

2. The method of claim 1, wherein the pathological intracranial pressure condition is selected from the group consisting of: moderate traumatic brain injury, severe traumatic brain injury, stroke, subarachnoid hemorrhage, idiopathic intracranial hypertension, pseudotumor cerebri, brain tumor, and cerebral malaria.

3. The method of claim 1, further comprising the step of updating the database of previously identified pathological intracranial pressure conditions with the diagnosis.

4. The method of claim 1, wherein the step of collecting raw data includes using transcranial Doppler.

5. The method of claim 1, wherein the reflected ultrasound waves are processed using Doppler waveform analysis.

6. The method of claim 1, wherein the structural features include at least one peak.

7. The method of claim 6, wherein the structural features further includes at least one sub-peak.

8. The method of claim 1, wherein the ultrasound transceiver is mounted within a headset device.

9. The method of claim 1, wherein the ultrasound transceiver is mounted within a hand-held portable device.

10. A non-invasive method for diagnosing mild traumatic brain injury in a patient, comprising the steps of:
providing stimuli to the patient;
non-invasively collecting raw data indicative of cerebral blood flow velocity from at least one blood vessel disposed within a cranial area of the patient;
converting the raw data into structural features including at least one peak and at least one sub-peak using a database of previously-validated cerebral blood flow velocity waveforms, wherein the structural features are indicative of a response rate of the subject to the stimuli;
classifying the structural features using a database of previously identified mild traumatic brain injury conditions; and
recommending a diagnosis based on the step of classifying.

11. The method of claim 11, wherein the step of collecting raw data includes using one of an MRI system, a CT scanner, pressure transducer, optical imaging and near-infrared imaging.

12. The method of claim 11, further comprising the step of updating the database of previously identified mild traumatic brain injury conditions with the diagnosis.

13. The method of claim 11, wherein the step of collecting raw data includes using transcranial Doppler.

14. The method of claim 11, wherein the step of collecting raw data includes transmitting and receiving reflections of ultrasound waves to a cranium of a patient using an ultrasound transceiver.

15. The method of claim 14, wherein the ultrasound transceiver is mounted within a headset device.

16. The method of claim 14, wherein the ultrasound transceiver is mounted within a hand-held portable device.

17. The method of claim 11, wherein the stimuli includes varying concentrations of arterial carbon dioxide (CO₂) of the patient.

18. The method of claim 17, wherein the concentrations of arterial carbon dioxide (CO₂) of the patient are varied by an action selected from the group of: exercising the patient, applying a leg cuff to the patient, and administering pharmaceuticals to the patient.

19. The method of claim 17, wherein the concentrations of arterial carbon dioxide (CO₂) of the patient are varied by an action selected from the group of: inducing the patient to hold his/her breath and inducing the patient to hyperventilate.

20. The method of claim 11, wherein the stimuli includes altering metabolism in the occipital lobe by exposing the patient to flashing lights.
